(19) United States
(12) Patent Application Publication
(10) Pub. No.: US 2009/0093441 A1
(43) Pub. Date: Apr. 9, 2009

(54) PREVENTING OR REDUCING OXIDATIVE STRESS OR OXIDATIVE CELL INJURY

(76) Inventors: Stephanie K. Lynch, Paris (FR); Maciej Turowski, Midland, MI (US); Wallace Yokoyama, Berkeley, CA (US)

Correspondence Address: The Dow Chemical Company, Intellectual Property Section, P.O. Box 1967, Midland, MI, 48641-1967, US

(21) Appl. No.: 11/874,663

(22) Filed: Oct. 18, 2007

Related U.S. Application Data
(60) Provisional application No. 60/853,381, filed on Oct. 20, 2006.

(51) Int. Cl. A61K 31/717; A61P 3/00

(52) U.S. Cl. 514/57

(57) ABSTRACT

A water-insoluble cellulose derivative, such as ethyl cellulose is useful for preventing or reducing oxidative stress or oxidative cell injury in tissues of an animal and in particular for influencing the level Stearoyl-CoA Desaturase-1 (SCD1) gene expression or ATP synthase mitochondrial F1 complex assembly factor 1 (ATPAF1) gene expression in non-adipose tissues of the animal.

This invention was made under a Cooperative Research And Development Agreement with the U.S. Department of Agriculture, number 58-3K95-5-1072.

### FIELD OF THE INVENTION

This invention relates to the prevention or reduction of oxidative stress or oxidative cell injury in tissues of an animal as well as to a medicament, pharmaceutical composition, food, food ingredient or supplement, or nutraceutical ingredient or supplement.

### BACKGROUND OF THE INVENTION

Oxidative stress is generally defined as an excess production of oxidizing agents in tissues. It is generally accepted in the medical sciences that oxidative stress can lead to cell injuries and eventually to cell death in such tissues.

Under normal physiological conditions, the use of oxygen by cells of aerobic organisms generates potentially deleterious reactive oxygen metabolites. A chronic state of oxidative stress exists in cells with an imbalance between prooxidants/oxidants and antioxidants. The amount of oxidative damage increases as an organism ages and is postulated to be a major causal factor of senescence (R S Sohal and R. Weindruck, Department of Biological Sciences, Southern Methodist University, Dallas, Tex. 75275, USA. Science, 1996 Jul. 5; 273(5271):59-63).

Over the past decade substantial scientific evidence in a wide variety of biomedical fields has implicated oxidative-free-radical injury and, in particular, excess production of reactive oxygen species (ROS), as primary factors causing cell death and tissue injury in a number of clinically important diseases, including cancer, central nervous system degenerative diseases, metabolic diseases, and ischemic cardiovascular diseases such as long-term complications of diabetes, arthritis, atherosclerosis and ischemia-reperfusion injury, as well as sun-induced skin damage and physical manifestations of aging. Well-known ROS are partially reduced O₂ derivatives, such as hydrogen peroxide, the hydroxyl radical, and the superoxide anion radical.

Alexander R W, Department of Medicine, Emory University School of Medicine, Atlanta, Ga., USA, “Transactions of the American Clinical and Climatological Association” (1998), 109 129-45 discloses that accumulating evidence provides a compelling case that one of the major pathophysiologic mechanisms involved in the pathogenesis of atherosclerosis is enhanced oxidative stress and that the most important manifestation of this altered redox state is the modulation of a set(s) of proinflammatory genes that are regulated directly or indirectly by reactive oxygen species. The author theorizes that hypercholesterolemia, hypertension, and diabetes mellitus related to age all activate similar redox-sensitive proinflammatory genes.

Large research efforts have been spent on finding medicinal antioxidants. As disclosed in U.S. Pat. No. 6,204,295, medicinal antioxidants are compounds that may be used for the prevention of tissue damage induced by lipid peroxidation (Haliwell, B., FASEB J. 1:358-364, 1987). U.S. Pat. No. 6,204,295 discloses that during lipid peroxidation free radicals interact with polyunsaturated fatty acids to form lipid peroxyl radicals, which produce lipid hydroperoxides and further lipid peroxyl radicals. This peroxidative cascade may eventually consume an essential part of the membrane lipid of a cell, which may lead to changes in membrane permeability and ultimately in cell death.

In view of the great importance of preventing or reducing oxidative stress or oxidative cell injury in tissues of individuals, particularly of human beings, large research efforts are not only spent on finding medicinal antioxidants, but a lot of research efforts are spent on studying the reactions of the individuals to oxidative stress or oxidative cell injury, for example on studying the molecular biological changes in tissues or body liquids of the individuals. Such molecular biological changes can serve as biomarkers for oxidative stress or oxidative cell injury.

Several studies have been published showing that high levels of reactive oxygen species (ROS) induce expression of the antioxidant enzyme SOD2. Superoxide dismutases (SOD) are important antioxidant enzymes responsible for the elimination of superoxide radical in the cells. The manganese-containing SOD (MnSOD or SOD2) is located in the mitochondria, where superoxide radical is constantly generated from the electron transport. For more that 30 years SOD was the only enzymatic system known to catalyse the elimination of superoxide (V. Niviere et al., Journal of Biological Inorganic Chemistry 9 (2): 119-123 March 2004, “Discovery of superoxide reductase: a historical perspective”). SOD has been found in almost all organism living in the presence of oxygen. SOD2 found in the mitochondria of organism from yeast to humans is taught to be a particularly important antioxidant defense (F. Archibald, PNAS 100 (18) 10141-10143, Sep. 2, 2003, “Oxygen toxicity and the health and survival of eukaryote cells: A new piece is added to the puzzle”).

T. Harju et al., Eur Respir J 2004; 24:765-771, “Manganese oxide superoxide dismutase is increased in the airways of smokers' lungs” disclose that oxidative stress is a key mechanism for smoking-induced chronic obstructive pulmonary disease. T. Harju et al. disclose that superoxide dismutases (SOD)s are the only enzymes capable of consuming superoxide radicals. The authors show that manganese superoxide dismutase (SOD2) is elevated in the alveolar epithelium of cigarette smokers, probably due to the increased oxidant burden in smokers' lungs.

Yumin Hu et al., Proc Amer Assos Cancer Res, Volume 46, 2005, “Expression of manganese superoxide dismutase (MnSOD) in human ovarian carcinoma and its role in cancer cell proliferation” disclose that they have performed a series of in vitro and in vivo studies to investigate the mechanistic link between MnSOD expression and ROS stress. An increase in superoxide generation by pharmacological interference of the mitochondrial respitory chain caused a rapid induction of MnSOD expression. C A Piantadosi et al, Free Radic. Biol. Med. 2006 Apr 15; 40(8):1332-9, “Carbon monoxide, oxidative stress, and mitochondrial permeability pore transition”, discuss that carbon monoxide induces manganese SOD (SOD2).

Since manganese SOD (SOD2) is an important antioxidant, it is generally not desirable to artificially suppress SOD2 expression. However, in view of the known mechanistic link between SOD2 expression and ROS, Applicants believe that SOD2 is a biomarker for ROS. An elevated level of expression or concentration of SOD2 in tissues of an animal is an indication of elevated levels of ROS. For example, it is well-known that oxidative stress or oxidative cell injury can be induced by elevated levels of ROS caused by high levels of fat in nutrition. Applicants believe that an increased level of expression or concentration of SOD2 is also induced by fat in nutrition. If a method of influencing the level of SOD2 expression or the concentration induced by ROS in tissues of animals can be found, for example, if a method of influencing the level of SOD2 expression or the concentration induced by fat in nutrition can be found, this would be a strong indication that this method would also affect or influence the level of ROS, for example induced by fat in nutrition, in tissues of animals.

Another protein that received great attention in biochemical science is tumor necrosis factor alpha (TNF-alpha, cachexin or cachectin). In medicine, TNF-alpha is an important cytokine involved in systemic inflammation and the acute phase response. TNF-alpha is released by white blood cells, endothelium and several other tissues in the course of damage, e.g. by infection (Wikipedia online). Since TNF-alpha plays a role in several diseases, a substantial amount of research has been conducted concerning TNF-alpha therapies and anti-TNF-alpha therapies. Because TNF-alpha exhibits anti tumor activity, research has been conducted to determine the protein's effectiveness against certain forms of cancers. Other research has focused upon inhibiting the effects of TNF-alpha in such diseases as Rheumatoid Arthritis, Crohn's Disease, AIDS, bacterial septic shock (caused by certain gram negative bacteria), and bacterial toxic shock (caused by superantigens) as well as in prevention of alloreactivity and graft rejection.

V. Verhasselt et al. discuss in Eur J. Immunol. 1998 November; 28(11):3886-90, “Oxidative stress up-regulates IL-8 and TNF-alpha synthesis by human dendritic [SP] cells” the effect of reactive oxygen intermediates, specifically H₂O₂ on human dendritic cells, a cell type which is critical for the initiation of the immune response. The authors observed that H₂O₂ stimulated the production of TNF-alpha by human dendritic cells in a dose-dependent manner.

Gordon W. Moe et al. published an article in Am J Physiol Heart Circ Physiol 287: H1813-H1820, 2004 with the title “In vivo TNF-α inhibition ameliorates cardiac mitochondrial dysfunction, oxidative stress, and apoptosis in experimental heart failure”.

Because TNF-alpha exhibits anti tumor activity, it may not desirable to artificially suppress TNF-alpha expression. However, in view of the disclosed connection between oxidative stress and TNF-alpha, Applicants believe that TNF-alpha is also a biomarker for oxidative stress. Applicants believe that an elevated level of expression or concentration of TNF-alpha in tissues of an animal is an indication of elevated levels of ROS. Applicants believe that an increased level of expression or concentration of TNF-alpha is also induced by fat in nutrition. If a method of influencing the level of TNF-alpha expression or concentration induced by ROS in tissues of animals can be found, for example, if a method of influencing the level of TNF-alpha expression or concentration induced by fat in nutrition can be found, this would be a strong indication that this method would also affect or influence the level of ROS, for example induced by fat in nutrition, in tissues of animals.

A third enzyme that received great attention in biochemical science is Stearoyl-CoA Desaturase-1 (SCD1). Studies have suggested that SCD1 appears to be an important metabolic control point, and decreasing the level of its expression could benefit the treatment of obesity, diabetes and other metabolic diseases. Stearoyl-Coenzyme A (CoA) Desaturase is a central lipogenic enzyme catalyzing the conversion of saturated acids, mainly palmitic acid and stearic acid, to monounsaturated fatty acids, mainly palmitoleate and oleate (J M Ntambi, M. Miyazaki, Department of Biochemistry and Nutritional Sciences, University of Wisconsin, Madison, USA: “Recent insights into Stearoyl-CoA Desaturase-1”, Curr Opin Lipidol. 2003 June; 14(3):255-61). J M Ntambi and M. Miyazaki disclose that mice that have a naturally occurring mutation in the SCD1 gene iso-form as well as a mouse model with a targeted disruption of the Stearoyl-CoA Desaturase gene-1 (SCD1−/−) have revealed the role of de-novo synthesized oleate and thus the physiological importance of SCD1 expression. It was found that mice with a disruption in the SCD1 gene (SCD1−/−) had increased energy expenditure, reduced body adiposity, increased insulin sensitivity, and are resistant to diet-induced obesity (“The role of Stearoyl-CoA Desaturase in Body Weight Regulation” by Agnieszka Dobrzyn and James M. Ntambi, TCM Vol. 14, No. 2, 2004)

SCD1 transcript has been found to be expressed in liver, lung, kidney, brain, stomach, muscle, adipose tissue, and skin. Fluorescent in situ hybridization showed that SCD1 expression in skin is restricted to the sebacieous glands, more specifically to the region containing mostly undifferentiated sebocytes, the bottom of the sebaceous gland (Ntambi et al., 1995; Ntambi et al., 1988; Zheng et al., 1999; Zheng et al., 2001).

In view of the substantial evidence that SCD1 is an important metabolic control point, it would be highly desirable to find a way of influencing the level of expression of one or more genes related to fat metabolism of tissues of an animal, preferably the expression of one or more genes inducing conversion of saturated fatty acids to monounsaturated fatty acids. It would be particularly desirable to find a way of reducing the level of SCD1 gene expression in tissues of individuals, particularly in non-adipose tissues.

Gene expression of ATP synthase, such as ATPAF1 (ATP synthase mitochondrial F1 complex assembly factor 1) gene expression, can also play an important role in preventing or reducing oxidative stress or oxidative cell injury in tissues of animals. ATP synthase is an enzyme that catalyzes the reaction of ATP synthesis and hydrolysis in the mitochondria. ATP (adenosine triphosphate) is used to provide energy for biochemical reactions, for example in the oxidation of fatty acids in the mitochondria in non-adipose tissues. Fatty acids are stored in the form of triacylglycerols primarily within adipocytes of adipose tissue. In response to energy demands, the fatty acids of stored triacylglycerols can be mobilized for use by non-adipose tissues. Fatty acids must be activated in the cytoplasm before being oxidized in the mitochondria. Activation is catalyzed by fatty acyl-CoA ligase (also called acyl-CoA synthetase or thiokinase). The net result of this activation process is the consumption of 2 molar equivalents of ATP.

Glucose and fatty acids are the ultimate sources of energy for animal cells. When glucose is scarce, fatty acids are mobilized for energy. A feature of insulin resistance is high concentrations of glucose and insulin in the blood, but a decreased transport of glucose into non-adipose tissues, such as peripheral tissues, despite high levels of insulin. Under these conditions fatty acids are converted to energy by mitochondria. While not wishing to be bound to the theory, Applicants believe that an elevated level of gene expression of ATPAF 1, a subunit of ATP synthase, is an indication of elevated oxidation of fatty acids in tissues, particularly in non-adipose tissues of animals, which can lead to oxidative stress or oxidative cell injury in such tissues. Accordingly, it would be desirable to find a way of influencing the level of expression of one or more genes related to mitochondrial oxidation pathways, and in particular of influencing the level of ATP synthase gene expression in tissues of animals, particularly in non-adipose tissues.

In view of the huge importance of preventing or reducing oxidative stress or oxidative cell injury in tissues of animals, particularly of human beings, it would be particularly desirable to find new methods which are useful for preventing or reducing oxidative stress or oxidative cell injury.

### SUMMARY OF THE INVENTION

It has surprisingly been found that administration of a water-insoluble cellulose derivative, such as ethyl cellulose, is useful for influencing the level of expression or the concentration of Stearoyl-CoA Desaturase-1 (SCD1) or ATP synthase mitochondrial F1 complex assembly factor 1 (ATPAF1) or both in tissues of animals.

It has also been surprisingly found that a water-insoluble cellulose derivative, such as ethyl cellulose, is useful for influencing the level of expression or the concentration of a superoxide dismutase, particularly of manganese superoxide dismutase (SOD2), or of tumor necrosis factor alpha (TNF-alpha) or both induced by reactive oxygen species in tissues of an animal.

Accordingly, one aspect of the present invention is a method of preventing or reducing oxidative stress or oxidative cell injury in a tissue of an animal, which method comprises the step of administering to the animal an effective amount of a water-insoluble cellulose derivative.

Another aspect of the present invention is a method of preventing or treating a disease of an organ of an animal caused or facilitated by oxidative stress or oxidative cell injury in said organ, which method comprises the step of administering to the animal an effective amount of a water-insoluble cellulose derivative.

Yet another aspect of the present invention is a method of influencing the level of expression of a gene related to fat metabolism of tissues of an animal, which method comprises the step of administering to the animal an effective amount of a water-insoluble cellulose derivative.

Yet another aspect of the present invention is a method of preventing or treating a disease of an organ of an animal caused or facilitated by Stearoyl-CoA Desaturase-1 (SCD1) gene expression or ATP synthase mitochondrial F1 complex assembly factor 1 (ATPAF1) gene expression or both, which method comprises the step of administering to the animal an effective amount of a water-insoluble cellulose derivative.

Yet another aspect of the present invention is a medicament, pharmaceutical composition, food, food ingredient or supplement, or nutraceutical ingredient or supplement which comprises an effective amount of a water-insoluble cellulose derivative for preventing or reducing oxidative stress or oxidative cell injury in a tissue of an animal.

Yet another aspect of the present invention is a medicament, pharmaceutical composition, food, food ingredient or supplement, or nutraceutical ingredient or supplement which comprises an effective amount of a water-insoluble cellulose derivative for preventing or treating a disease of an organ of an animal caused or facilitated by oxidative stress or oxidative cell injury in said organ.

Yet another aspect of the present invention is a medicament, pharmaceutical composition, food, food ingredient or supplement, or nutraceutical ingredient or supplement which comprises an effective amount of a water-insoluble cellulose derivative for influencing the level of expression of a gene related to fat metabolism of tissues of an animal.

Yet another aspect of the present invention is a medicament, pharmaceutical composition, food, food ingredient or supplement, or nutraceutical ingredient or supplement which comprises an effective amount of a water-insoluble cellulose derivative for preventing or treating a disease of an organ of an animal caused or facilitated by Stearoyl-CoA Desaturase-1 (SCD1) gene expression or ATP synthase mitochondrial F1 complex assembly factor 1 (ATPAF1) gene expression or both.

Yet another aspect of the present invention is the use of a water insoluble cellulose derivative for the manufacture of a medicament, pharmaceutical composition, food, food ingredient or supplement, or nutraceutical ingredient or supplement to prevent or reduce oxidative stress or oxidative cell injury in a tissue of an animal.

Yet another aspect of the present invention is the use of a water insoluble cellulose derivative for the manufacture of a medicament, pharmaceutical composition, food, food ingredient or supplement, or nutraceutical ingredient or supplement to prevent or treat a disease of an organ of an animal caused or facilitated by oxidative stress or oxidative cell injury in said organ.

Yet another aspect of the present invention is the use of a water insoluble cellulose derivative for the manufacture of a medicament, pharmaceutical composition, food, food ingredient or supplement, or nutraceutical ingredient or supplement to influence the level of expression of a gene related to fat metabolism of tissues of an animal.

Yet another aspect of the present invention is the use of a water insoluble cellulose derivative for the manufacture of a medicament, pharmaceutical composition, food, food ingredient or supplement, or nutraceutical ingredient or supplement to prevent or treat a disease of an organ of an animal caused or facilitated by Stearoyl-CoA Desaturase-1 (SCD1) gene expression or ATP synthase mitochondrial F1 complex assembly factor 1 (ATPAF1) gene expression or both.

Yet another aspect of the present invention is a water-insoluble cellulose derivative as a medicament for the prevention or reduction of oxidative stress or oxidative cell injury in a tissue of an animal.

Yet another aspect of the present invention is a water-insoluble cellulose derivative as a medicament for the prevention or treatment of a disease of an organ of an animal caused or facilitated by oxidative stress or oxidative cell injury in said organ.

Yet another aspect of the present invention is a water-insoluble cellulose derivative as a medicament for influencing the level of expression of a gene related to fat metabolism of a tissue of an animal.

Yet another aspect of the present invention is a water-insoluble cellulose derivative as a medicament for the prevention or reduction of a disease of an organ of an animal caused or facilitated by Stearoyl-CoA Desaturase-1 (SCD1) gene expression or ATP synthase mitochondrial F1 complex assembly factor 1 (ATPAF1) gene expression or both.

### DETAILED DESCRIPTION OF THE INVENTION

Since oxidative stress is generally defined as an excess production of oxidizing agents in tissues, the term “a method of preventing or reducing oxidative stress or oxidative cell injury” as used herein includes a method of preventing or reducing an excess production of oxidizing agents in tissues, in particular excess production of reactive oxygen species (ROS).

The term “a method of preventing or reducing oxidative stress or oxidative cell injury” as used herein includes any treatment that delays the development of oxidative stress or oxidative cell injury in time or in severity or that reduces the severity of developing or developed oxidative stress or oxidative cell injury.

The term “influencing the level of expression of a gene by administration of a water-insoluble cellulose derivative” as used herein means that a body tissue, such as blood, has a different, generally a lower, expression of said gene after the intake of a water-insoluble cellulose derivative by an individual, as compared to the expression of said gene after the intake of a non-effective material such as unmodified cellulose itself. The term “influencing the level of expression of a gene” is not limited to the direct regulation of gene expression but also includes the indirect influence on gene expression, for example by influencing the conditions or metabolites in a body tissue which lead to a different, generally lower gene expression.

More specifically, the term “influencing the level of Stearoyl-CoA Desaturase-1 (SCD1) gene expression or ATP synthase mitochondrial F1 complex assembly factor 1 (ATPAF1) gene expression” as used herein means that a body tissue, such as blood, has a different, preferably a lower, SCD1 gene expression or ATPAF1 gene expression after the intake of a water-insoluble cellulose derivative by an individual, as compared to the SCD1 gene expression or ATPAF1 gene expression after the intake of unmodified cellulose itself.

The term “influencing the level of expression or the concentration of a superoxide dismutase, particularly of manganese superoxide dismutase (SOD2), or the level of expression or the concentration of tumor necrosis factor alpha (TNF-alpha)” as used herein means that a body tissue, such as blood, has a different, preferably a lower, level of expression or concentration of a superoxide dismutase, particularly SOD2, or of TNF-alpha after the intake of a water-insoluble cellulose derivative by an individual, as compared to the level of expression or the concentration of a superoxide dismutase, particularly SOD2, or of TNF-alpha after the intake of a non-effective material such as unmodified cellulose itself.

The term “preventing or treating a disease of an organ of an animal caused or facilitated by SCD1 gene expression or ATPAF1 gene expression or both” as used herein means that conditions in an organ of an animal are prevented or treated which involve SCD1 or ATPAF1 gene expression, particularly that conditions in an organ of an animal are prevented or treated which would lead to elevated SCD1 or ATPAF1 gene expression without prevention or treatment. SCD1 and/or ATPAF1 gene expression are believed to be bio-markers for conditions which can lead a related disease of an organ of an animal. The term “animal” relates to any animals including human beings. Preferred animals are mammals. The term “mammal” refers to any animal classified as a mammal, including human beings, domestic and farm animals, such as cows, nonhuman primates, zoo animals, sports animals, such as horses, or pet animals, such as dogs and cats.

The term “tissue” relates to an organization of a plurality of similar cells with varying amounts and kinds of nonliving, intercellular substance between them, such as epithelial tissues, connective tissues, for example fluid connective tissues like blood, muscle tissues or nervous tissues.

The term “organ” relates to an organization of several different kinds of tissues so arranged that together they can perform a special function.

The cellulose derivatives which are useful in the present invention are water-insoluble. The term “cellulose derivative” does not include unmodified cellulose itself which also tends to be water-insoluble. Experiments conducted by the Applicants have surprisingly shown that water-insoluble cellulose derivatives have a significantly different effect on Stearoyl-CoA Desaturase-1 (SCD1) gene expression and/or ATPF1 gene expression in tissues of animals than unmodified cellulose. Experiments conducted by the Applicants have also shown that water-insoluble cellulose derivatives have a different effect on the level of expression or the concentration of manganese superoxide dismutase and/or tumor necrosis factor alpha in tissues of animals than unmodified cellulose.

The term “water-insoluble” as used herein means that the cellulose derivative has a solubility in water of less than 2 grams, preferably less than 1 gram, in 100 grams of distilled water at 25° C. and 1 atmosphere.

Preferred cellulose derivatives for use in the present invention are water-insoluble cellulose ethers, particularly ethyl cellulose, propyl cellulose or butyl cellulose. Other useful water insoluble cellulose derivatives are cellulose derivatives which have been chemically, preferably hydrophobically, modified to provide water insolubility. Chemical modification can be achieved with hydrophobic long chain branched or non-branched alkyl, arylalkyl or alkylaryl groups. “Long chain” typically means at least 5, more typically at least 10, particulary at least 12 carbon atoms. Other type of water-insoluble cellulose are crosslinked cellulose, when various crosslinking agents are used. Chemically modified, including the hydrophobically modified, water-insoluble cellulose derivatives are known in the art. They are useful provided that they have a solubility in water of less than 2 grams, preferably less than 1 gram, in 100 grams of distilled water at 25° C. and 1 atmosphere. The most preferred cellulose derivative is ethyl cellulose. The ethyl cellulose preferably has an ethoxyl substitution of from 40 to 55 percent, more preferably from 43 to 53 percent, most preferably from 44 to 51 percent. The percent ethoxyl substitution is based on the weight of the substituted product and determined according to a Zeisel gas chromatographic technique as described in ASTM D4794-94 (2003). The molecular weight of the ethyl cellulose is expressed as the viscosity of a 5 weight percent solution of the ethyl cellulose measured at 25° C. in a mixture of 80 volume percent toluene and 20 volume percent ethanol. The ethyl cellulose concentration is based on the total weight of toluene, ethanol and ethyl cellulose. The viscosity is measured using Ubbelohde tubes as outlined in ASTM D914-00 and as further described in ASTM D446-04, which is referenced in ASTM D914-00. The ethyl cellulose generally has a viscosity of up to 400 mPa·s, preferably up to 300 mPa·s, more preferably up to 100 mPa·s, measured as a 5 weight percent solution at 25° C. in a mixture of 80 volume percent toluene and 20 volume percent ethanol. The preferred ethyl celluloses are premium grades ETHOCEL ethyl cellulose which are commercially available from The Dow Chemical Company of Midland, Mich. Combinations of two or more water-insoluble cellulose derivatives are also useful.

Preferably the water-insoluble cellulose derivative has an average particle size of less than 0. 1 millimeter, more preferably less than 0.05 millimeter, most preferably less than 0.02 millimeter. Preferably the water-insoluble cellulose derivative is exposed to an edible fat or oil before being administered to an individual so that the cellulose derivative imbibes the fat or oil. Advantageously the water-insoluble cellulose derivative is exposed to an excess of the fat or oil at about 40 to 60° C.

Applicants have surprisingly found that administration of a water-insoluble cellulose derivative is useful for influencing the level of expression of one or more genes related to fat metabolism of tissues of an animal, particularly for influencing the level of expression of one or more genes for the conversion of saturated fatty acids to monounsaturated fatty acids and/or for influencing the level of expression of one or more genes related to mitochondrial oxidation pathways, and in particular for influencing, particularly reducing, the level of Stearoyl-CoA Desaturase-1 (SCD1) gene expression and/or ATPF1 gene expression in tissues, particularly in non-adipose tissues, such as the liver, pancreas, lungs, kidneys, brain, stomach or in muscles. Applicants have found that the water-insoluble cellulose derivatives influence the level of expression of genes responsible for saturated fat desaturation and/or mitochondrial oxidation pathways. Without wanting to be bound to the theory, Applicants believe that the hydrophobic residue of the water-insoluble cellulose derivatives contributes to the regulation and normalization of the fat metabolism by water-insoluble cellulose derivatives.

Since SCD1 catalyzes the conversion of saturated fatty acids, particularly palmitic acid and stearic acid, to monounsaturated fatty acids, particularly palmitoleate and oleate, Applicants conclude that elevated SCD1 expression, herein designated as SCD1 gene over-expression, in tissues particularly in non-adipose tissues, is an indication of an elevated concentration of saturated fatty acids in these tissues. By the term “gene over-expression” as used herein is meant the level of expression of a gene which is higher than the normal level of expression of the gene in healthy animals. For example, obesity is typically accompanied by SCD1 gene over-expression, i.e. by a higher level of SCD1 gene expression than in animals of normal weight.

Furthermore, Applicants conclude that elevated SCD 1 gene expression in non-adipose tissues is an indication of oxidative stress in cells or even oxidative cell injury in these tissues. While the adipocytes in adipose tissue have a unique capacity to store excess fatty acids in the form of triglycerides in lipid droplets, non-adipose tissues, such as peripheral tissues, have a limited capacity for storage of lipids. Laura L. Listenberger et al., PNAS, Mar. 18, 2003, vol. 100, no. 6, 3077-3082, “Triglyceride accumulation protects against fatty acid-induced lipotoxicity”, suggests that accumulation of excess lipid in non-adipose tissues leads to cell disfunction and/or cell death, a phenomenon known as lipotoxicity. These authors suggest that lipotoxicity from accumulation of long chain fatty acids is specific for saturated fatty acids and that this selectivity for saturated fatty acids has been attributed to signaling molecules in response to saturated but not unsaturated fatty acids, including reactive oxygen species generation (ROS).

Applicants have compared SCD1 gene expression in tissues of pairs of animals after administration of a) a high-fat diet comprising microcrystalline cellulose to control animals and b) the same high fat diet to the other animals, except that microcrystalline cellulose is replaced with a water-insoluble cellulose derivative to the other animals. Applicants have found that animals fed with the same fat and caloric diet as control animals show a significantly lower SCD1 gene expression in tissues, particularly in non-adipose tissues, when the diet is supplemented with a water-insoluble cellulose derivative. The lower SCD1 expression is an indication that administering a water-insoluble cellulose derivative is useful for preventing or reducing oxidative stress or oxidative cell injury in tissues, particularly in non-adipose tissues. Without wanting to be bound to the theory, Applicants conclude from the lower SCD1 expression that the concentration of saturated fats is not high enough to increase SCD1 expression, although the animals ingest the same amount of fat as the control animals. Applicants conclude that the lower SCD1 expression in such tissues of animals, whose diet is supplemented with a water-insoluble cellulose derivative, is sufficient to convert saturated fats into unsaturated fats and into triglyceride storage. The observed lower SCD1 expression in non-adipose tissues of animals, whose diet is supplemented with a water-insoluble cellulose derivative but who ingest the same amount of fat as control animals, leads the Applicants to conclude that water-insoluble cellulose derivatives prevent or reduce accumulation of excess saturated fats in non-adipose tissues and therefore are useful for preventing or reducing oxidative stress or oxidative cell injury in such tissues which could ultimately lead to cell disfunction and/or cell death.

Applicants have surprisingly found that administration of a water-insoluble cellulose derivative is also useful for influencing, particularly reducing, the level of ATPF1 gene expression in tissues, particularly in non-adipose tissues, of an animal.

Based on the findings described in more detail above, Applicants conclude that influencing the level of SCD1 and/or ATPF1 gene expression contributes to the prevention or reduction of oxidative stress or oxidative cell injury in tissues of an animal, and accordingly to the prevention or treatment of a disease of an organ of an animal caused or facilitated by oxidative stress or oxidative cell injury of said organ. The present invention is particularly useful for the prevention or reduction of oxidative stress or oxidative cell injury and the diseases related thereto which is induced by fat in nutrition, particularly by an imbalanced nutrition with a high fat content.

The above-discussed finding is confirmed by the finding of the Applicants that administration of a water-insoluble cellulose derivative is also useful for influencing the level of gene expression of a superoxide dismutase (SOD), particularly manganese-containing SOD (MnSOD or SOD2) and/or of tumor necrosis factor alpha (TNF-alpha) in tissues of animals. Applicants have compared SOD2 and TNF-alpha gene expression in tissues of pairs of animals after administration of a) a high-fat diet comprising microcrystalline cellulose to control animals and b) the same high fat diet to the other animals, except that microcrystalline cellulose is replaced with a water-insoluble cellulose derivative. Applicants have found that animals fed with the same fat and caloric diet as control animals show a significantly lower SOD2 and TNF-alpha gene expression in tissues, particularly in non-adipose tissues, when the diet is supplemented with a water-insoluble cellulose derivative. Without wanting to bound by the theory, Applicants believe that the lower SOD2 and TNF-alpha gene expressions are an indication that less reactive oxygen species (ROS) are induced in tissues due to the fat in nutrition and accordingly less SOD2 and TNF-alpha is induced in response to ROS when the diet is supplemented with a water-insoluble cellulose derivative. The observed lower SOD2 and TNF-alpha gene expressions in non-adipose tissues of animals, whose diet is supplemented with a water-insoluble cellulose derivative but who ingest the same amount of fat as control animals, leads the Applicants to also to conclude that water-insoluble cellulose derivatives are useful for preventing or reducing oxidative stress or oxidative cell injury in such tissues which could ultimately lead to cell disfunction and/or cell death.

The present invention is particularly useful for the prevention or reduction of oxidative stress or oxidative cell injury and the diseases related thereto which are induced by fat in nutrition, particularly by an imbalanced nutrition with a high fat content.

The water-insoluble cellulose derivative can be administered or consumed in or as a medicament, pharmaceutical composition, food, food ingredient or supplement, or nutraceutical ingredient or supplement. The medicament, pharmaceutical composition, food, food ingredient or supplement, or nutraceutical ingredient or supplement can be solid or liquid. The desired time period of administering the water-insoluble cellulose derivative can vary depending on the amount of water-insoluble cellulose derivative consumed, the general health of the animal, the level of activity of the animal and related factors. It may be advisable to administer or consume the water-insoluble cellulose derivative as long as nutrition with a high fat content is consumed. Generally administration of at least 1 to 12 weeks, preferably 3 to 8 weeks is recommended.

It is to be understood that the duration and daily dosages of administration as disclosed herein are general ranges and may vary depending on various factors, such as the specific cellulose derivative, the weight, age and health condition of the individual, and the like. It is advisable to follow the prescriptions or advices of medical doctors or nutrition specialists when consuming the water-insoluble cellulose derivatives.

According to the present invention the water-insoluble cellulose derivatives are preferably used for preparing food, a food ingredient or supplement, or a nutraceutical ingredient or supplement which comprises from 0.5 to 20 weight percent, more preferably from 2 to 15 weight percent, most preferably from 4 to 12 weight percentage of one or more water-insoluble cellulose derivatives. The given weight percentages relate to the total amount of the water-insoluble cellulose derivatives. The amount administered is preferably in the range of from 1 to 10 percent of the total daily weight of the diet of the individual on a dry weight basis. Preferably, the water-insoluble cellulose derivative is administered or consumed in sufficient amounts throughout the day, rather than in a single dose or amount. When the water-insoluble cellulose derivatives are administered or consumed in combination with water, the water-insoluble cellulose derivatives will generally not suffer from the “mouth feel” compliance issues, which are sometimes created by water-soluble cellulose derivatives due to their tendency to form slimy viscous solutions with water.

Although the water-insoluble cellulose derivatives are preferably administered in combination with food or as foodstuff, alternatively they can be administered as an aqueous suspension or in powder form or as pharmaceutical or nutraceutical compositions. Pharmaceutical or nutraceutical compositions containing water-insoluble cellulose derivatives can be administered with an acceptable carrier in a pharmaceutical or nutraceutical unit dosage form. Pharmaceutically acceptable carriers include tableting excipients, gelatin capsules, or carriers such as a polyethylene glycol or a natural gel. Pharmaceutical or nutraceutical unit dosage forms include tablets, capsules, gelatin capsules, pre-measured powders and pre-measured solutions. Hence, the water-insoluble cellulose derivatives may be formulated as tablets, granules, capsules and suspensions.

Regardless whether the water-insoluble cellulose derivative is administered as an aqueous suspension or in powder form, as a medicament, pharmaceutical or nutraceutical composition or is combined with other food ingredients, the amount of administered water-insoluble cellulose derivative is generally in the range of from 10 to 300 milligrams of water-insoluble cellulose derivative per pound of mammal body weight per day. About 2 g to about 30 g, preferably about 3 g to about 15 g of water-insoluble cellulose derivative are ingested daily by a large mammal such as a human.

While the method of administration or consumption may vary, the water-insoluble cellulose derivatives are preferably ingested by a human as a food ingredient of his or her daily diet. The water-insoluble cellulose derivatives can be combined with a liquid vehicle, such as water, milk, vegetable oil, juice and the like, or with an ingestible solid or semi-solid foodstuff, such as “veggie” burgers, spreads or bakery products.

A number of foodstuffs are generally compatible with water-insoluble cellulose derivatives. For example, a water-insoluble cellulose derivative may be mixed into foods such as milk shakes, milk shake mixes, breakfast drinks, juices, flavored drinks, flavored drink mixes, yogurts, puddings, ice creams, ice milks, frostings, frozen yogurts, cheesecake fillings, candy bars, including “health bars” such as granola and fruit bars, gums, hard candy, mayonnaise, pastry fillings such as fruit fillings or cream fillings, cereals, breads, stuffing, dressings and instant potato mixes. An effective amount of water-insoluble cellulose derivatives can also be used as a fat-substitute or fat-supplement in salad dressings, frostings, margarines, soups, sauces, gravies, mayonnaises, mustards and other spreads. Therefore, “food ingredients,” as the term is used herein, includes those ingredients commonly employed in recipes for the above foodstuffs, including flour, oatmeal, fruits, milk, eggs, starch, soy protein, sugar, sugar syrups, vegetable oils, butter or emulsifying agents such as lecithin. Colorings and flavorings may be added as may be appropriate to add to the attractiveness of the foodstuff.

The water-insoluble cellulose derivative can also be administered to domestic and farm animals, such as cows, nonhuman primates, zoo animals, sports animals, such as horses, or pet animals, such as dogs and cats, in a known manner in or as a medicament, pharmaceutical composition, food, food ingredient or supplement, or nutraceutical ingredient or supplement. A preferred way of administration is the incorporation of a water-insoluble cellulose derivative in the pet feed or other animal feed for preventing or reducing oxidative stress or oxidative cell injury in a tissue of the animal and/or for preventing or treating a disease of an organ of an animal caused or facilitated by oxidative stress or oxidative cell injury in said organ, such as mitochondrial and/or metabolic diseases, such as insulin resistance, diabetes, or hypercholesterolemia and/or hypertension related to diabetes, particularly of cats or dogs.

Since the present invention is also useful for preventing or reducing oxidative stress or oxidative cell injury, particularly oxidative stress or oxidative cell injury induced by fat in nutrition, the present invention is also useful for preventing or treating a disease that is caused or facilitated by oxidative stress or oxidative cell injury of said organ. Such diseases are numerous. For example, the present invention is useful for preventing or treating liver diseases, such as hepatitis; cancer; central nervous system degenerative diseases, mitochondrial and/or metabolic diseases, such as insulin resistance, Type II Diabetes, or hypercholesterolemia and/or hypertension related to diabetes, atherosclerosis; ischemic injuries, such as cardiac ischemic injury; inflammatory diseases and auto-immune diseases, such as inflammatory bowel disease, rheumatoid arthritis, or Crohn's Disease; cardiovascular diseases, such as coronary heart disease or post-ischemic arrhythmias; neurological diseases, such as Alzheimer's, stroke, bovine Spongiform Encephalopathy (BSE; Mad Cow Disease); Creutzfeld Jacob Disease (CJD; human variant of BSE); muscle damage; sun-induced skin damage, physical manifestations of aging, or for the treatment of AIDS.

The present invention is particularly useful for preventing or treating diseases that are associated by the skilled persons with the expression, particularly over-expression of Stearoyl-CoA Desaturase-1 in tissues of animals, including mitochondrial and/or metabolic diseases, such as insulin resistance, Type II Diabetes or hypercholesterolemia and/or hypertension related to diabetes.

The water-insoluble cellulose derivative is optionally used in combination with water-soluble or water-insoluble naturally occurring polymers or derivatives thereof, such as gum arabic, xanthan gum or derivatives thereof, gum karaya, gum tragacanth, gum ghatti, guar gum or derivatives thereof, exudate gums, seaweed gums, seed gums, microbial gums, carrageenan, dextran, gelatin, alginates, pectins, starches or derivatives thereof, chitosans or other polysaccharides, preferably beta-glucans, galactomannans, hemicelluloses, psyllium, guar, xanthan, microcrystalline cellulose, amorphous cellulose or chitosan.

In some embodiments of the present invention it is particularly beneficial to use or administer a water-insoluble cellulose derivative in combination with a water-soluble cellulose derivative. Useful amounts of combinations of one or more water-insoluble cellulose derivatives and one or more water-soluble cellulose derivatives and useful ways for administration, consumption or inclusion of such combinations in a medicament, pharmaceutical composition, food, food ingredient or supplement, or nutraceutical ingredient or supplement are generally the same as those described above for the water-insoluble cellulose derivatives alone.

The water-soluble cellulose derivatives have a solubility in water of at least 2 grams, preferably at least 3 grams, more preferably at least 5 grams in 100 grams of distilled water at 25° C. and 1 atmosphere. Preferred water-soluble cellulose derivatives are water-soluble cellulose esters and cellulose ethers. Preferred cellulose ethers are water-soluble carboxy-C₁-C₃-alkyl celluloses, such as carboxymethyl celluloses; water-soluble carboxy-C₁-C₃-alkyl hydroxy-C₁-C₃-alkyl celluloses, such as carboxymethyl hydroxyethyl celluloses; water-soluble C₁-C₃-alkyl celluloses, such as methylcelluloses; water-soluble C₁-C₃-alkyl hydroxy-C₁-₃-alkyl celluloses, such as hydroxyethyl methylcelluloses, hydroxypropyl methylcelluloses or ethyl hydroxyethyl celluloses; water-soluble hydroxy-C₁-₃-alkyl celluloses, such as hydroxyethyl celluloses or hydroxypropyl celluloses; water-soluble mixed hydroxy-C₁-C₃-alkyl celluloses, such as hydroxyethyl hydroxypropyl celluloses, water-soluble mixed C₁-C₃-alkyl celluloses, such as methyl ethyl celluloses, or water-soluble alkoxy hydroxyethyl hydroxypropyl celluloses, the alkoxy group being straight-chain or branched and containing 2 to 8 carbon atoms. The more preferred cellulose ethers are methylcellulose, methyl ethyl cellulose, hydroxyethyl cellulose, hydroxypropyl cellulose, hydroxyethyl methylcellulose, hydroxypropyl methylcellulose, and carboxymethyl cellulose, which are classified as water-soluble cellulose ethers by the skilled artisans. The most preferred water-soluble cellulose ethers are methylcelluloses with a methyl molar substitution DS_{methoxyl} of from 0.5 to 3.0, preferably from 1 to 2.5, and hydroxypropyl methylcelluloses with a DS_{methoxyl} of from 0.9 to 2.2, preferably from 1.1 to 2.0, and a MS_{hydroxypropoxyl} of from 0.02 to 2.0, preferably from 0. 1 to 1.2. The methoxyl content of methyl cellulose can be determined according to ASTM method D 1347-72 (reapproved 1995). The methoxyl and hydroxypropoxyl content of hydroxypropyl methylcellulose can be determined by ASTM method D-2363-79 (reapproved 1989). Methyl celluloses and hydroxypropyl methylcelluloses, such as K100M, K4M, K1M, F220M, F4M and J4M hydroxypropyl methylcellulose are commercially available from The Dow Chemical Company). The water-soluble cellulose derivative generally has a viscosity of from 5 to 2,000,000 cps (=mPa·s), preferably from 50 cps to 200,000 cps, more preferably fromt 75 to 100,000 cps, in particular from 1,000 to 50,000 cps, measured as a two weight percent aqueous solution at 20 degrees Celsius. The viscosity can be measured in a rotational viscometer.

The present invention is further illustrated by the following examples which are not to be construed to limit the scope of the invention. Unless otherwise mentioned, all parts and percentages are by weight.

### EXAMPLE 1

An animal study was conducted with male golden Syrian hamsters with a starting body weight of 70-90 grams (Sasco strain, Charles River, Wilmington, Mass.) in each of the two diets specified below. The animal study was approved by the Animal Care and Use Committee, Western Regional Research Center, USDA, Albany, Calif.

Significance at 95% level is listed for the data in the examples below (p<0.05). Since the data are the results obtained on biological, living systems, variation within the same group of animals is to be expected.

The effect of administering an ethyl cellulose to hamsters was tested. The ethyl cellulose used in Example 1 is commercially available from The Dow Chemical Company under the trademark ETHOCEL Standard Premium 10 FP. FP stand for “fine particles” grade ethyl cellulose. It has an ethoxyl content of 48.0-49.5 percent and a viscosity of about 10 mPa·s, measured as a 5 weight percent solution at 25° C. in a mixture of 80 volume percent toluene and 20 volume percent ethanol using a Brookfield viscometer.

The male Syrian golden hamsters were divided into two groups. One of the groups was called “treatment group” and was fed a high-fat treatment diet and water ad libitum, while the other group was called “control group” and was fed high-fat control diet and water ad libitum. Both groups counted 10 hamsters each. These groups were fed for a period of eight consecutive weeks.

A water-insoluble cellulose ether was present at 5 weight percent level in the treatment diet. In case this treatment diet, water-insoluble cellulose ether was first suspended in liquefied fat fraction of the diet, before mixing with the powdered fractions of the diet. For this treatment diet, a 1000 g of either of the complete high-fat treatment diets contained 150 g of butter fat, 50 g of corn oil, 200 g of casein, 499 g of corn starch, 3 g of DL methionine, 3 g of choline bitartrate, 35 g of a mineral mixture, 10 g of a vitamin mixture and 50 g of ETHOCEL Standard Premium 10 FP “fine” grade ethyl cellulose.

The control diet had exactly same composition as treatment diet, with the only exception that the water-insoluble cellulose derivative was replaced by same amount of microcrystalline cellulose (MCC), mixed into powdered components of the diet during the control diet preparation.

After the hamsters had been fed the diets for eight consecutive weeks, the livers were taken out from four or more animals of the treatment group and four or more animals of the control group on a random basis. The sacrificed hamsters of the treatment group are designated in Table 5 below as HF-EC-1, HF-EC-2, HF-EC-3 and HF-EC-4. The sacrificed hamsters of the control group are designated in Table 5 below as HF-Control-1 and HF-Control-2, HF-Control-3 and HF-Control-4.

The gene expressions for Stearoyl-CoA Desaturase-1 (SCD1), tumor necrosis factor alpha (TNF-alpha) and manganese superoxide dismutase (SOD2) were determined by mRNA Extraction and Analysis. Total mRNA (messenger ribonucleic acid) was extracted, purified, and reverse transcribed according to Bartley and Ishida (2002). The teaching of Bartley, G. E. and Ishida, B. K. (2002) Digital Fruit Ripening: Data Mining in the TIGR Tomato Gene Index. Plant Mol. Biol. Rep. 20: 115-130, is included herein by reference.

cDNAs resulting from reverse transcription of the above total mRNAs were diluted 10 fold and 1 microliter aliquots were used in real-time PCR reactions with specific primers for the genes having a length of 20-24 bases as decribed further below and SYBR Green Supermix (BIO-RAD) according to the manufacturer's protocols with the following changes: 1. Reactions were performed in 25-microliter total volume in triplicate reactions 2. An MX3000P (Stratagene) instrument was used to perform the PCR. PCR conditions were 5 min at 95° C. followed by 40 cycles of incubation at 94° C.×15 s, 55 to 60° C.×1 min and72° C.×30 s. The following primers were used:

| | | |
|---|---|---|
| SCD-1: | GCCACCTGGCTGGTGAACAGTG | (forward), |
| | GGTGGTAGTTGTGGAAGCCCTCG | (reverse); |
| SOD2: | TAAGGAGCAAGGTCGCTTACAGA | (forward), |
| | CTCCCAGTTGATTACATTCCAAAT | (reverse); |
| TNF-alpha: | GCCGCATTGCTGTGTCCTACG | (forward), |
| | GGCACTGAGTCGGTCACCTTTCT | (reverse); |
| Actin: | ACGTCGACATCCGCAAAGACCTC | (forward), |
| | TGATCTCCTTCTGCATCCGGTCA | (reverse). |

Primer efficiencies were determined using dilution curves of cDNA. Relative quantitation was performed by normalization to the actin transcript as in Livak, K. J. and Schmittgen, T. D. (2001). The teaching of Livak, K. J. and Schmittgen, T. D. (2001), Analysis of relative gene expression data using real-time quantitative PCR and the 2^{-ΔΔC}T Method. Methods. 25: 402-408, is incorporated herein by reference. Negative controls to determine the extent of DNA contamination were carried out with identical concentrations of total mRNAs that were not reverse transcribed. A negative control was run for some of the primer sets. In each case the no-reverse transcription control signal was achieved after 5 or more cycles than the samples that were transcribed.

The SCD1, TNF-alpha and SOD2 gene expression of the hamster HF-EC-1 was compared with the SCD1, TNF-alpha and SOD2 gene expression of the hamsters HF-Control-1 and HF-Control-2. The ratios for the gene expressions HF-EC-1/HF-Control-1 and HF-EC-1/HF-Control-2 are listed in Table 1 below. The ratios for the SCD1, TNF-alpha and SOD2 gene expression were determined for other pairs of hamsters as listed in Table 1 below.

For comparative purposes, the effect of a water-soluble hydroxypropyl methyl cellulose (HPMC) on SCD1, TNF-alpha and SOD2 gene expression was also studied. The same experiments as described above were conducted, except that HPMC was used in the high fat diet (HF-HPMC) instead of ethyl cellulose. In the control diet HPMC was replaced with microcrystalline cellulose. The HPMC had a methoxyl content of 19-24 percent, a hydroxypropoxyl content of 7-12 percent and a viscosity of about 100,000 mPa·s, measured as a 2 wt. % aqueous solution at 20° C., and is commercially available from The Dow Chemical Company under the Trademark METHOCEL K100M hypromellose.

The results are listed in Table 1 below. The values in Table 1 for each animal pair and each gene are an average of triplicate measurements. The mean and standard error of the mean (SEM) values are given. It is understood that the numbers expressed in the Table 1 are relative to control, i.e. if the number is lower than 1 then the expression of a particular gene is lower in the hamsters from the treatment group than in the hamsters from the control group, and vice versa.

TABLE 1

| Animal pairs, ratio of | | | |
|---|---|---|---|
| gene expression | SCD1 | TNF-alpha | SOD2 |
| HF-EC-1/HF-Control-1 | 0.29 | 0.64 | 0.58 |
| HF-EC-1/HF-Control-2 | 0.26 | 0.48 | 0.61 |
| HF-EC-2/HF-Control-1 | 0.24 | 0.88 | 0.58 |
| HF-EC-2/HF-Control-2 | 0.22 | 0.63 | 0.63 |
| HF-EC-3/HF-Control-3 | 0.31 | 1.1 | 0.61 |
| HF-EC-3/HF-Control-4 | 0.32 | 0.84 | 0.51 |
| HF-EC-4/HF-Control-3 | 0.29 | 1.4 | 1.1 |
| HF-EC-4/HF-Control-4 | 0.30 | 1.0 | 0.90 |
| Mean | 0.28 | 0.87 | 0.69 |
| standard error of the mean (SEM) | 0.01 | 0.10 | 0.07 |
| HF-HPMC-1/HF-Control-1 * | 0.39 | 1.31 | 0.85 |
| HF-HPMC-1/HF-Control-2 * | 0.35 | 0.93 | 0.92 |
| HF-HPMC-2/HF-Control-1 * | 0.22 | 0.87 | 0.69 |
| HF-HPMC-2/HF-Control-2 * | 0.25 | 0.62 | 0.69 |
| HF-HPMC-3/HF-Control-3 * | 0.16 | Not assessed | 0.80 |
| HF-HPMC-3/HF-Control-4 * | 0.16 | Not assessed | 1.4 ** |
| HF-HPMC-4/HF-Control-3 * | 0.28 | Not assessed | 0.53 |
| HF-HPMC-4/HF-Control-4 * | 0.28 | Not assessed | 0.88 |
| Mean | 0.26 | 0.93 | 0.77 |
| standard error of the mean (SEM) | 0.03 | 0.14 | 0.05 |

* Not within the scope of the present invention, but not prior art
** Eliminated for calculating Mean and SEM based on “Standard Practice for Dealing With Outlying Observations” ASTM E 178-80. A statistical outlier analysis was done using the Grubb's analysis [Grubbs, Frank (February 1969), Procedures for Detecting Outlying Observations in Samples, Technometrics, Vol. 11, No. 1, pp. 1-21 and http://www.itl.nist.gov/div898/handbook/eda/section3/eda35h.htm].

While the data show some variation within the same group of animals, this is to be expected since the results are obtained on biological, living systems. Nevertheless, the data show a clear trend. The administration of a water-insoluble cellulose derivate, such as ethyl cellulose, has the most prominent effect on Stearoyl Co-A Desaturase-1 (SCD1). Although fed with the identical high fat diet, the hamsters that were additionally fed with ethyl cellulose (instead of microcrystalline cellulose) had a significantly lower SCD1 gene expression. The TNF-alpha and SOD2 gene expression were also lower in the animals that were fed a diet containing ethyl cellulose than in control animals that were fed a diet that did not comprise a water-insoluble cellulose derivative. The reduced SCD1, TNF-alpha and SOD2 gene expression are a clear indication for the usefulness of a water-insoluble cellulose derivate, such as ethyl cellulose, preventing or reducing oxidative stress or oxidative cell injury in tissues of an animal. The effect of ethyl cellulose is at least as good or sometimes even better than the effect of HPMC which has been evaluated for comparative purposes.

### EXAMPLE 2

The procedure for Example 1 was repeated, except that for the measurements the animals were grouped diffently and the ATP synthase mitochondrial F1 complex assembly factor 1 (ATPAF1) gene expression was measured. The following specific primer for

ATPAF1 was used:
ACTCCTGGCCAGACTCTAATACA (forward);
CACAGGCAGAGTTCAGGGAGTAG (reverse).

The results are listed in Table 2 below. The mean and standard error of the mean (SEM) values are given.

TABLE 2

| Animal pairs, ratio of | |
|---|---|
| gene expression | ATPAF1 |
| HF-EC-3/HF-Control-4 | 0.77 |
| HF-EC-3/HF-Control-1 | 0.92 |
| HF-EC-4/HF-Control-4 | 0.79 |
| HF-EC-4/HF-Control-1 | 0.96 |
| HF-EC-5/HF-Control-5 | 0.77 |
| HF-EC-5/HF-Control-6 | 0.61 |
| HF-EC-6/HF-Control-5 | 0.93 |
| HF-EC-6/HF-Control-6 | 0.67 |
| Mean | 0.80 |
| standard error of the mean (SEM) | 0.04 |
| HF-HPMC-3/HF-Control-4* | 0.45 |
| HF-HPMC-3/HF-Control-1* | 0.57 |
| HF-EC-2/HF-Control-4* | 0.68 |
| HF-EC-2/HF-Control-1* | 0.89 |
| HF-EC-5/HF-Control-5* | 0.78 |
| HF-EC-5/HF-Control-6* | 0.59 |
| HF-EC-4/HF-Control-5* | 0.50 |
| HF-EC-4/HF-Control-6* | 0.38 |
| Mean | 0.61 |
| standard error of the mean (SEM) | 0.06 |

*Not within the scope of the present invention, but not prior art

The higher levels of synthase mitochondrial F1 complex assembly factor 1 (ATPAF1) in animals fed the HF-Control diet than in animals fed the HF-EC and HF-HPMC diets is evidence for a higher level of fat oxidation for energy production in the animals fed with the HF diet.

### EXAMPLE 3

An animal study was conducted with male golden Syrian hamsters with a starting body weight of 50-60 grams (LVG strain, Charles River, Wilmington, Mass.) in each of the diets specified below. The animal study was approved by the Animal Care and Use Committee, Western Regional Research Center, USDA, Albany, Calif. The effect of administering ethyl cellulose to hamsters was tested as previously described in Example 1. The ethyl cellulose used in Example 3 was ETHOCEL Standard Premium 10 “fine” grade ethyl cellulose. It is commercially available from The Dow Chemical Company and has an ethoxyl content of 48.0-49.5 percent and a viscosity of about 10 mPa·s, measured as a 5 weight percent solution at 25° C. in a mixture of 80 volume percent toluene and 20 volume percent ethanol using a Brookfield viscometer.

The male Syrian golden hamsters were divided into three groups. Two groups were called “treatment group” and was fed diets containing “EC dry” and “EC fat”. One group was called “control group” and was fed a diet consisting of microcrystalline cellulose (MCC). Each group consisted of approximately 10 hamsters each. These groups were fed for a period of three consecutive weeks.

Treatment Group 1: EC Dry

This treatment group was fed an EC treatment diet. 1000 g of the dry EC treatment diet contained 80 g of butter fat, 100 g of corn oil, and 20 g of fish oil and 1 g of cholesterol, 200 g of casein, 498 g of corn starch, 3 g of DL methionine, 3 g of choline bitartrate, 35 g of a mineral mixture, 10 g of a vitamin mixture and 50 g of ETHOCEL Standard Premium 10 “fine” grade ethyl cellulose.

Treatment Group 2: EC Fat

The EC fat diet for Treatment Group 2 was the same as the diet for Treatment Group 1, except that the 50 g of ETHOCEL Standard Premium 10 ethyl cellulose was dispersed in the diet fat portion at 50° C. during the diets preparation.

Control Group: MCC

The control diet had exactly the same composition as the treatment diet, with the only exception that the ethyl cellulose was replaced by the same amount of microcrystalline cellulose (MCC), mixed into powdered components of diet during the control diet preparation.

After the hamsters had been fed the diets for three consecutive weeks, plasma was obtained and the livers were removed from both the treatment groups and control group.

### Quantitative RT-PCR Analysis SCD-1 and SOD2 in Hamster Livers

The gene expressions for manganese superoxide dismutase (SOD2) and Stearoyl-CoA Desaturase-1 (SCD-1) were determined by mRNA extraction and analysis as described in Example 1.

The SCD1 and SOD2 gene expression of the hamsters in “EC dry” and “EC fat” groups was compared with SCD1 and SOD2 gene expression of the hamsters control MCC group. The ratios for the gene expression are listed in Table 3 below. The mean and standard error of the mean (SEM) values are given. It is understood that the numbers expressed in the Table 3 are relative to control, i.e. if the number is lower than 1 then the expression of a particular gene is lower in hamsters from the treatment group than in the hamsters from the control group, and vice versa.

TABLE 3

| Ratio of Gene Expression | SCD1 Mean (SEM) | SOD2 Mean (SEM) |
|---|---|---|
| EC dry/control MCC | 0.48 (0.15) | 1.29 (0.09) |
| EC fat/control MCC | 0.96 (0.23) | 1.17 (0.06) |

While the data show some variation within the same group of animals, this is to be expected since the results are obtained on biological, living systems. Nevertheless, the data show a clear trend. The administration of water-insoluble cellulose derivate, such as ethyl cellulose, has the most prominent effect on Stearoyl Co-A Desaturase-1 (SCD1). Even though the diet was only three weeks the hamsters fed with the ethyl cellulose diet instead of microcrystalline cellulose had a significantly lower SCD1 gene expression. Interestingly, the SOD2 gene expression was elevated in animals that were fed a diet containing ethyl cellulose for three weeks compared to the control animals. This is different than the results of SOD2 gene expression observed in Example 1. In the other animal studies the diets were administered for eight weeks compared to three weeks in this study. Nevertheless the reduced SCD1 gene expression is a clear indication for the usefulness of water-insoluble cellulose derivate, such as ethyl cellulose, for preventing or reducing oxidative stress or oxidative cell injury in tissues of an animal.

### Analysis of SOD Activity in Hamster Plasma

Hamster EDTA plasma samples were assayed for SOD activity based on the reaction of a tetrazolium salt with the superoxide radicals generated by xanthine oxidase and hypoxanthine. Due to the fact that extracellular SOD (SOD3) accounts for the majority of the SOD activity in plasma, total SOD activity was measured for all three types of SOD.

Plasma samples were diluted 10-fold with sample buffer provided in the Superoxide Dismutase assay kit, Cayman Chemical (Ann Arbor, Mich.) prior to analysis. The dilution factor was pre-determined to ensure the enzymatic activity fell within the standard curve range. SOD activity analysis was performed based on the procedure provided with the kit with minor modifications in the order the reagents were added. In brief, 10 μL of standards or diluted plasma was added to the designated wells followed by the addition of 20 μL of diluted xanthine oxidase to all the wells. The reaction was initiated by adding 200 μL of the diluted radical detector. Because this assay measures the kinetics of the reaction, the last reagent should be added as quickly as possible (preferably using multi-channel pipette). After brief shaking of the plate to mix, both kinetic and end-point measurements at 450 nm were performed for 20 minutes at room temperature. The kinetic measurement of each sample provides information of the linearity of the reaction kinetics regime. The end-point measurement was used to generate a standard curve based on linearized rate (LR; LR for Std B=Abs_{450 nm} Std A/Abs_{450 nm} Std B) and SOD activities of the standards. The SOD activity of the unknown sample was calculated based on the linear regression of the standard curve and the following equations:

$$S O D ( U \text{/} mL ) = [ ( \frac{sampleLR - y\_intercept}{slope} ) \times \frac{0.23}{0.01} ] \times 10 \qquad ( 1 )$$

Total superoxide dismutase (SOD, including SOD1, SOD2, and SOD3) levels in hamster plasma samples of this animal study are summarized in Table 7. The SOD level of each sample was then normalized with the albumin concentration of the same sample prior to further data analysis. Outlier detection was performed using multivariate analysis with Mahalanobis diagnostic. The normalized SOD levels of the hamsters in different diet groups were analyzed after the outliers were excluded. After normalization the SOD levels in the different diet groups were shown not to be statistically different from the MCC control group. The mean SOD level of all animals in this study coincides with the mean SOD level of MCC group. The SOD activity is similar to the SOD2 gene expression data.

TABLE 7

| Diet | [SOD]* | Ratio |
|---|---|---|
| EC dry | 13.7 ± 2.2 | 0.95 |
| EC fat | 14.6 ± 2.5 | 1.01 |
| MCC | 14.5 ± 2.7 | — |

*mean ± standard deviation

Collectively, the results in Example 3 are an indication that water-insoluble cellulose derivatives such as ethyl cellulose are useful for preventing or reducing oxidative stress or oxidative cell injury in tissues of an animal.

What is claimed is:

1. A method of preventing or reducing oxidative stress or oxidative cell injury in a tissue of an animal, comprising the step of administering to the animal an effective amount of a water-insoluble cellulose derivative.

2. The method of claim 1 wherein oxidative stress or oxidative cell injury induced by fat in nutrition is prevented or reduced.

3. The method of claim 1 wherein oxidative stress or oxidative cell injury in the liver, pancreas, lungs, kidneys, brain, stomach or in muscles of a mammal is prevented or reduced.

4. The method of claim 1 wherein the level of expression or the concentration of manganese superoxide dismutase (SOD2) or of tumor necrosis factor alpha (TNF-alpha) or of both, induced by fat in nutrition, is influenced in a tissue of an animal.

5. The method of claim 1 for influencing the level of Stearoyl-CoA Desaturase-1 (SCD1) gene expression or ATP synthase mitochondrial F1 complex assembly factor 1 (ATPAF1) gene expression or both.

6. A method of preventing or treating a disease of an organ of an animal caused or facilitated by oxidative stress or oxidative cell injury in said organ, comprising the step of administering to the animal an effective amount of a water-insoluble cellulose derivative.

7. The method of claim 6 for preventing or treating cancer, liver diseases, central nervous system degenerative diseases, auto-immune diseases, metabolic diseases, mitochondrial diseases, ischemic injuries, inflammatory diseases, cardiovascular diseases, neurological diseases, muscle damage, sun-induced skin damage, physical manifestations of aging or for the treatment of AIDS.

8. A method of influencing the level of expression of a gene related to fat metabolism of a tissue of an animal, the method comprising the step of administering to the animal an effective amount of a water-insoluble cellulose derivative.

9. The method of claim 8 wherein the level of expression of a gene related to the fat metabolism of non-adipose tissues is influenced.

10. The method of claim 8 wherein the level of expression of a gene for the conversion of saturated fatty acids to monounsaturated fatty acids is influenced.

11. The method of claim 8 wherein the level of expression of a gene related to mitochondrial oxidation pathways is influenced.

12. The method of claim 8 wherein the level of Stearoyl-CoA Desaturase-1 (SCD1) gene expression or ATP synthase mitochondrial F1 complex assembly factor 1 (ATPAF1) gene expression or both is influenced.

13. The method of claim 12 wherein the level of Stearoyl-CoA Desaturase-1 (SCD1) gene expression or ATP synthase mitochondrial F1 complex assembly factor 1 (ATPAF1) gene expression or both in the liver, pancreas, lungs, kidneys, & brain, stomach or in muscles of a mammal is influenced.

14. A method of preventing or treating a disease of an organ of an animal caused or facilitated by Stearoyl-CoA Desaturase-1 (SCD1) gene expression or ATP synthase mitochondrial F1 complex assembly factor 1 (ATPAF1) gene expression or both, comprising the step of administering to the animal an effective amount of a water-insoluble cellulose derivative.

15. The method of claim 14 wherein a mitochondrial or metabolic disease is prevented or treated.

16. The method of claim 1 wherein the water-insoluble cellulose derivative is ethyl cellulose.

17. The method of claim 1 wherein from 10 to 300 milligrams of water-insoluble cellulose derivative per pound of animal body weight is administered per day in the form of a medicament, pharmaceutical composition, food or food supplement or nutraceutical ingredient or supplement.

18. A medicament, pharmaceutical composition, food, food ingredient or supplement, or nutraceutical ingredient or supplement comprising an effective amount of a water-insoluble cellulose derivative.

19. The medicament, pharmaceutical composition, food, food ingredient or supplement, or nutraceutical ingredient or supplement of claim 18 wherein the water-insoluble cellulose derivative is ethyl cellulose.


SEQUENCE LISTING (10 sequences)

SEQ ID NO: 1 - DNA, 22 bp, Artificial
  Synthetic Construct
gccacctggctggtgaacagtg

SEQ ID NO: 2 - DNA, 23 bp, Artificial
  Synthetic Construct
ggtggtagttgtggaagccctcg

SEQ ID NO: 3 - DNA, 23 bp, Artificial
  Synthetic Construct
taaggagcaaggtcgcttacaga

SEQ ID NO: 4 - DNA, 24 bp, Artificial
  Synthetic Construct
ctcccagttgattacattccaaat

SEQ ID NO: 5 - DNA, 21 bp, Artificial
  Synthetic Construct
gccgcattgctgtgtcctacg

SEQ ID NO: 6 - DNA, 23 bp, Artificial
  Synthetic Construct
ggcactgagtcggtcacctttct

SEQ ID NO: 7 - DNA, 23 bp, Artificial
  Synthetic Construct
acgtcgacatccgcaaagacctc

SEQ ID NO: 8 - DNA, 23 bp, Artificial
  Synthetic Construct
tgatctccttctgcatccggtca

SEQ ID NO: 9 - DNA, 23 bp, Artificial
  Synthetic Construct
actcctggccagactctaataca

SEQ ID NO: 10 - DNA, 23 bp, Artificial
  Synthetic Construct
cacaggcagagttcagggagtag